(19) United States
(12) Patent Application Publication
(10) Pub. No.: US 2023/0338329 A1
(43) Pub. Date: Oct. 26, 2023

(54) CANNABINOID EMULSION COMPOSITION AND METHOD OF MANUFACTURING

(71) Applicant: Douglas Gore, Carlsbad, CA (US)

(72) Inventor: Douglas Gore, Carlsbad, CA (US)

(21) Appl. No.: 17/727,532

(22) Filed: Apr. 22, 2022

(51) Int. Cl. A61K 31/352; A61K 9/107; A61K 47/14; A61K 31/05; A61K 31/355; A61K 31/122

(52) U.S. Cl. CPC A61K 31/352; A61K 9/1075; A61K 47/14; A61K 31/05; A61K 31/355; A61K 31/122

(57) ABSTRACT

A Cannabinoid emulsion composition includes Vitamin E TPGS, a Cannabinoid, and a Carrier Oil, in the absence of water, heated and mixed in such proportions that the composition forms nano or micro sized micelles when ingested. The composition may be formed via a first step of heating the Vitamin E TPGS, a Cannabinoid, and a Carrier Oil to 30-100 degrees Celsius (86-212 degrees Fahrenheit), while mixing, for 10-20 minutes, then allowed to cool, while mixing, until an emulsion is formed.

### BACKGROUND OF THE INVENTION

### Field of the Invention

This invention relates generally to Cannabinoid compositions, and more particularly to a lipid-based oil-in-oil (o/o) Cannabinoid emulsion composition and method of manufacturing.

### Description of Related Art

The word “Cannabis” refers to a genus of flowering plants. Plants of genus Cannabis include several species, including Cannabis sativa, Cannabis indica, and Cannabis ruderalis. Plants from genus Cannabis are useful a wide variety of medicinal purposes, as well as recreational activities, etc., discussed further below.

According to some accounts, Cannabis is composed of at least 483 known chemical compounds, which include Cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, Terpenes, and non-Cannabinoid phenols.

Cannabinoids and Terpenes are of particular interest for research and commercialization. Most extractions of Cannabis plant matter aim to extract Cannabinoids, particularly tetrahydrocannabinol (THC) and Cannabidiol (CBD). THC is useful for relieving pain, treating glaucoma, relieving nausea, and a wide variety of other uses. CBD is useful for seizure disorder (epilepsy), anxiety, chronic pain, inflammation, and a muscle disorder called Dystonia, as well as Parkinson's disease, Crohn's disease, and many other conditions. Usually, Cannabinoids are extracted from the Cannabis plant as part of a crude mixture. Cannabinoids and Terpenes are separated by different extraction processes to produce purified Cannabinoids and purified Terpenes.

Most current methods of administration of Cannabinoids fail to take full advantage of Cannabinoid properties. For example, burning plant matter and inhaling the vapor does not allow for selection of certain Cannabinoids and Terpenes for their certain desired benefit. One can choose a plant with certain known properties, e.g., THC content, but there is still little to no control over selecting individual Cannabinoids. Inhaling smoke also leads to many harmful and toxic compounds introduced into the body.

When Cannabinoids (CBD, CBN, THC, Terpenes, etc.) are extracted from the cannabis plant or other plant matter, it takes an oil-based form. Like any oil, it is hydrophobic; it will not dissolve in water. Cannabinoids, Terpenes, Oil based (lipophilic) Vitamins/Antioxidants, Essential Oils and Botanical Oils are extremely hydrophobic or lipophilic and show low bioavialability when administered through oral route. Having a poor aqueous solubility and low dissolution rate limits the oral bioavailability. Oral administration is an appealing due to the simplicity, convenience, high patient compliance, suitability for chronic therapy and reduced costs for physicians and industry. However, there are still numerous inherent challenges hampering the effective delivery of drugs, such as low water solubility, limited permeability through the gastrointestinal tract, poor stability against enzymes and hydrolysis effect, which lead to poor absorption and bioavailability. Poor water solubility and/or poor permeability remain as the major obstacles for lipophilic ingredients and therapeutic drugs to exert maximum activity.

Lipid based drug delivery systems in their simplest form involve dissolving the lipophilic active ingredients in a lipid carrier. These simplest formulations tend to suffer from high food effects, as the lipid carrier needs bile salts to become properly dispersed. Most emulsions are thermodynamically unstable and thus require the presence of surfactant(s). Although oil-in-water (o/w) and water-in-oil (w/o) emulsions have found widespread use across a number of fields, the presence of water in these systems limits the type of functionalities that can be used.

The prior art teaches a variety of a water soluble Cannabinoid formulations. Examples are shown in Levy, U.S. Pat. No. 10,722,490, and also 10,568,865 (assigned to Canopy Grown Corporation, of Ontario, Canada), which were granted Jul. 28, 2020 and Feb. 25, 2020, respectively. These patents disclose a water soluble Cannabinoid formulation that includes one or more purified Cannabinoids, one or more purified Terpenes, and TPGS, in water.

Kuhrts, U.S. Pat. No. 10,328,111, teaches a phytoCannabinoid emulsion formulation that includes: a solution of water and propylene glycol; a phytoCannabinoid oil; a non-ionic surfactant. The weight ratio of phytoCannabinoid content to non-ionic surfactant can be from 1:10,000 to 1:5.

Bromley, U.S. Pat. No. 9,861,611, claims a formulation of water-soluble derivatives of Vitamin E and soft gel compositions, that include a water-soluble Vitamin E derivative mixture (of a particular composition); a non-polar ingredient; a sugar fatty acid ester; and a binder.

Berl, U.S. 2020/0138772, teaches an emulsion that includes CBD, THC, and an emulsifier such as Vitamin E.

Schaneville, U.S. Pat. No. 10,632,164, claims a narrow emulsion that includes (in water), the following: an extract of cannabis or Hemp; a thickening agent (selected from a list of such agents); a flavoring agent (selected from a list of such agents); a taste masking agent (selected from a list of such agents); a plasticizer (selected from a list of such agents); a sweetener; a film forming agent; a stabilizing agent; and a binder.

Goskonda, U.S. Ser. No. 10/265,293 and U.S. Pat. No. 8,222,292, teach oral Cannabinoid formulations, including an aqueous-based oral dronabinol solution, that are stable at room or refrigerated temperatures and may possess improved in vivo absorption profiles with faster onset and lower inter-subject variability.

Eades, U.S. 2020/0093787, describes a variety of compositions for administering Cannabinoid, including edible forms of Cannabinoids. The compositions include Vitamin E, glycerin, and lecithin enriched with phospholipids. Phospholipids and waxes may be used to control the onset timing of cannabis drug effects.

Schwarz, U.S. 2020/0022386, teaches a method of preparation of beverages, containing poorly water soluble Cannabinoids, by two-stage dilution of the self-nanoemulsifying concentrate, and composition of the concentrate for preparation of such beverages.

Friedman, U.S. 2019/0298683, teaches self-emulsifying, high concentration and high dose Cannabinoid compositions and formulations, to improve administration of Cannabinoids and standardized marijuana extracts to patients.

Goskonda, U.S. 2019/0192428, teaches oral Cannabinoid formulations, including an aqueous-based oral dronabinol solution, that are stable at room or refrigerated temperatures and may possess improved in vivo absorption profiles with faster onset and lower inter-subject variability.

There exists a need for new Cannabinoid and Terpene formulations. In particular, there exists a need for Cannabinoid and Terpene formulations with increased permeability into the bloodstream. Additionally, there exists a need to increase absorption and bioavailability of Cannabinoids (CBD, CBN, THC, etc.), Terpenes, Essential Oils and other Lipophilic Vitamins and Lipophilic Antioxidants together or individually in stable workable o/o emulsions for topical and ingestible products.

### SUMMARY OF THE INVENTION

The present invention teaches certain benefits in construction and use which give rise to the objectives described below.

The present invention provides a Cannabinoid emulsion composition comprising Vitamin E TPGS, a Cannabinoid, and a Carrier Oil, in the absence of water, heated and mixed in such proportions that the composition forms nano or micro sized micelles when ingested.

In one embodiment, the invention teaches a method for forming an emulsion containing a Cannabinoid. The method has a first step of heating Carrier Oil and a Cannabinoid in separate containers on a hot plate to a temperature between 30-100 degrees Celsius (86-212 degrees Fahrenheit), while mixing, then adding the heated Carrier Oil to the heated Cannabinoid and mixing to form a first composition. The method has a second step of heating Vitamin E TPGS on the hot plate to a temperature between 30-100 degrees Celsius (86-212 degrees Fahrenheit), while mixing, then adding the first composition to the heated Vitamin E TPGS, while mixing, to form a second composition. A final step of the method is mixing the second composition at 30-100 degrees Celsius (86-212 degrees Fahrenheit) for 10-20 minutes, then turning off the heat, while mixing, until the second composition is at room temperature and an emulsion is formed.

A primary objective of the present invention is to provide a Cannabinoid emulsion composition and a method for forming an emulsion containing a Cannabinoid having advantages not taught by the prior art.

Another objective is to provide a Cannabinoid emulsion composition that is stable and provides superior bioavailability of the Cannabinoid to the user.

Another objective is to provide a Cannabinoid emulsion composition formed via a superior method for forming an emulsion that includes heating ingredients separately, then mixing to form a novel composition.

Another objective is to provide a method of forming a Cannabinoid emulsion composition that forms micelles when applied to a user or ingested.

A further objective is to provide a method of forming a Cannabinoid emulsion composition that is suitable for ingesting, or which may be applied topically to a user.

Other features and advantages of the present invention will become apparent from the following more detailed description of the invention.

### DETAILED DESCRIPTION OF THE INVENTION

The invention includes a Cannabinoid emulsion composition, and in particular an oil-in-oil (o/o) Cannabinoid emulsion composition and method of manufacture.

Cannabinoid Emulsion Composition

The Cannabinoid emulsion composition includes a Cannabinoid which is emulsified with Vitamin E TPGS, without the use of water. As discussed in more detail below, an emulsion of this nature is not typically suitable and stable; however, we have found through extensive experimentation that it can be made stable with the inclusion of suitable proportions of a Carrier Oil (excipient). Various methods of manufacturing these compositions are discussed in greater detail below.

In some embodiments, the Cannabinoid emulsion composition includes a Cannabinoid and a Carrier Oil (excipient) that is selected to have high amounts of long chain triglycerides (LCT), which are emulsified with Vitamin E TPGS (emulsifier non-ionic antioxidant surfactant) using a novel manufacturing process described in greater detail below. The selected Carrier Oil(s) act as a lipid stabilizer, as discussed in greater detail below.

In some embodiments, the Carrier Oil includes at least 5% (by volume) of polyunsaturated fatty acids and monounsaturated fatty acids, in some embodiments having polyunsaturated fatty acids and monounsaturated fatty acids in an amount greater than 50%. The Cannabinoid emulsion composition may further include oil-based forms of Vitamins A, C, D, E, and K, as well as antioxidants and flavoring agents. Examples of these Vitamins within the context of this disclosure include: Vitamin A existing in three forms: retinol, retinal, and retinoic acid. Forms of Vitamin C might include magnesium ascorbyl phosphate,L-ascorbic acid, Tetrahexyldecyl ascorbate, ascorbyl palmitate, ascorbyl glucosamine, and ascorbyl tetraisopalmitate. Vitamin D3, and Vitamin E existing in eight basic forms of the entire fat soluble Vitamin E molecule, which are either synthetically or naturally derived. The most typical forms are d-alpha-tocopherol, d-alpha-tocopherol acetate, dl-alpha tocopherol, and dl-alpha tocopherol acetate. The “d” prefix in front of the “alpha” indicates that the product was derived from natural sources, such as vegetable oils or wheat germ; the “dl” prefix indicates that the Vitamin was created from a synthetic base. Vitamin E tocopherol, the term tocopherol refers to any naturally occurring or synthetic form of Vitamin E, and can refer to a single compound or a mixture. Examples of tocopherols include, for example, a-tocopherol, D-a-tocopherol, β-tocopherol, γ-tocopherol, and δ-tocopherol. Vitamin K is a family of naphthoquinone compounds comprising K1 (phylloquinone) and several forms of K2 (MKs, menaquinones).

Absence of Water

While prior art formulations of this nature typically include water, this formulation does not include water. For purposes of this application, any reference to the formulation to the absence of water is defined to mean that water is less than 1% of the final composition (by volume), as some amount of water may be present without departing from the present invention.

In various embodiments, the composition can be used as a topical or taken orally depending on the combination of ingredients. Being that this invention does not use any water, only Vitamin E TPGS, Carrier Oil(s), and lipophilic ingredients, products such as tinctures, serums, creams, oral gels/capsules, gummies, beverages, etc., can be created. This type of emulsion composition produces nano or micro sized droplets (micelles) when they come into contact with an aqueous solution such as in the mouth or stomach, which may enhance/increase permeation across cellular membranes throughout the digestive system. When applied topically there is an increase in permeation within the epidermis and dermis, increasing the topical absorption/permeability and bioavailability through different mechanisms (discussed in greater detail below). These products may be used to treat a variety of skin conditions, including acne vulgaris, allergic contact dermatitis, asteatotic, dermatitis, atopic dermatitis, hidradenitis suppurative, Kaposi sarcoma, pruritus, psoriasis, skin cancer, cutaneous manifestations of systemic sclerosis, to name only a few of the many potential applications. “Bioavailability” as used in this application is defined as the percentage of the substance absorbed into the bloodstream after it is fully processed throughout the body.

Vitamin E TPGS

D-α-Tocopherol polyethylene glycol 1000 succinate, also known as Vitamin E TPGS (TPGS). TPGS 1000 (d-alpha-tocopheryl polyethylene glycol 1000 succinate) is a water-soluble form of Vitamin E. TPGS 1000 is a widely used form of Vitamin E TPGS. TPGS 1000 comprised of hydrophilic polar (water-soluble) head and lipophilic (water-insoluble) alkyl tail. Due to its surface active properties, it can be used as a solubilizer, an emulsifier, and as a vehicle for lipid-based drug delivery formulations. TPGS may be used as an effective oral absorption enhancer for improving the bioavailability of poorly absorbed drugs, and is FDA approved as a water-soluble Vitamin E nutritional supplement and drug delivery vehicle.

The ingredients with Vitamin E TPGS might include the following: Carrier Oil(s), Essential Oil(s) over 130+, Cannabinoid(s) (there are at least 113 identified types of Cannabinoids in cannabis), CBD isolate, Full Spectrum CBD, Broad Spectrum CBD, THC Distillate, THC Isolate, more than 150 different Terpene(s), Vitamins A, D, E, K, and antioxidants such as: Ascorbyl Palmitate, Carotenes, Ubiquinol (Coenzyme Qio), a flavoring agent may or may not be used. Vitamin E TPGS is a synthetic amphiphile that undergoes enzymatic cleavage to deliver the lipophilic antioxidant, α-tocopherol (Vitamin E) to cell membranes.

TPGS is also a precipitation inhibitor, and modulates P-glycoprotein (P-gp) efflux transport via P-gp ATPase inhibition mechanism and acts as a potent excipient that promotes the efficiency of delivery and the therapeutic effect of drugs. Vitamin E TPGS is a more potent P-gp inhibitor than many associated excipients with surfactant properties, such as Pluronic P85, Cremophor EL, Tween 80, and PEG 300. TPGS can solubilize water-soluble and lipophilic molecules, forming various types of micelles and increasing the solubility and bioavailability of cannibinoids, Terpenes, botanical oils, and drugs like cyclosporines, taxanes, steroids, and antibiotics.

In another example, the emulsifier is a TPGS analog. TPGS analog refer to compounds, other than TPGS, that are similar to a parent TPGS compound, but differ slightly in composition, for example, by the variation, addition or removal of an atom, one or more units (e.g., methylene unit(s)-(CH2)n) or one or more functional groups. TPGS analogs include Vitamin E derived surfactants, including Vitamin E PEG diesters, such as, but not limited to, tocopherol polyethylene glycol sebacate (PTS), tocopherol polyethylene glycol dodecanodioate (PTD), tocopherol polyethylene glycol suberate (PTSr), tocopherol polyethylene glycol azelate (PTAz), and polyoxyethanyl tocotrienyl sebacate (PTrienS) as well as other PEG derivatives of Vitamin E.

Further suitable examples of Vitamin-E derived emulsifiers include, but are not limited to, polyethylene glycol (PEG) derivatives of tocopherol, such as tocopherol polyethylene glycol diesters (TPGD). A preferred emulsifier is tocopherol polyethylene glycol succinate (TPGS). TPGS analogs, TPGS homologs, and TPGS derivatives are also suitable. Other examples of emulsifiers include tocopherol sebacate polyethylene glycol, tocopherol dodecanodioate polyethylene glycol, tocopherol suberate polyethylene glycol, tocopherol azelate polyethylene glycol, tocopherol citraconate polyethylene glycol, tocopherol methyl citraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol, among others.

Antioxidants

The emulsions disclosed herein can also contain an antioxidant. The antioxidant can be present in the continuous phase and/or the dispersed phase. Suitable examples of antioxidants include, but are not limited to, a phenolic compound, a plant extract, or a sulphur-containing compound. In certain examples disclosed herein the antioxidant can be ascorbic acid or a salt thereof, e.g., sodium ascorbate. In other examples, the antioxidant can be Vitamin E, tocopherols, lipid soluble derivatives of more polar antioxidants such as ascobyl fatty acid esters (e.g., ascobyl palmitate), plant extracts (e.g., rosemary, sage and oregano oils, green tea extract), algal extracts, and synthetic antioxidants (e.g., BHT, TBHQ, ethoxyquin, alkyl gallates, hydroquinones, tocotrienols), etc. Other types of Lipophilic antioxidants might include Vitamin A, carotenoids, docosahexaenoic acid (DHA) and eicosapentaenoic acid (EPA), flavonoids, resveratrol, retinyl palmitate, trans-lycopene, retinyl stearate, and nitrones. The Cannabinoid emulsion composition may further include CoQ₁₀ (aka ubiquinone), which is a powerful anti-oxidant.

For purposes of this application, the term “Cannabinoid” is defined to include one or more of the known Cannabinoid molecules, including but not limited to THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, THC isolate, and equivalent materials. Cannabinoids are insoluble in water; but they are soluble in non-polar solvents (e.g. fat and oil). Cannabinoids require a suitable emulsification process to overcome the immiscibility of Cannabinoids in water.

In addition to these base ingredients, we add, in some embodiments, Essential Oil(s), Terpenes, and/or equivalent molecules as well. Examples of Terpenes within the context of this disclosure include, but are not limited to, the following: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin. Within the context of this disclosure, the term Terpene includes the .alpha.-(alpha), .beta.-(beta), .gamma.-(gamma), oxo-, isomers, or any combinations thereof.

In one embodiment, the purified Terpene is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Cannabinoids and Terpenes are highly lipophilic molecules (log P 6-7) with very low aqueous solubility (2-10 μg/mL), that are susceptible to degradation, especially in solution, via the action of light and temperature as well as via auto-oxidation. Formulations can play a crucial role in increasing the solubility and physicochemical stability.

As used herein, the term “purified” means extracted, isolated, and/or separated from other compounds, formulations, compositions, matter, and/or mass. In one embodiment, the term “purified” refers to a Cannabinoid that is separated from the plant matter from which it was derived. In one embodiment, the term “purified” refers to a Cannabinoid (a “purified Cannabinoid”) that is separated from other Cannabinoids that were present in the plant matter from which it was derived. In one embodiment, the term “purified” refers to a Cannabinoid (a “purified Cannabinoid”) that is separated from Terpenes that were present in the plant matter from which it was derived. In one embodiment, the term “purified” refers to a Cannabinoid (a “purified Cannabinoid”) that is separated from secondary compounds that were present in the plant matter from which it was derived. In one embodiment, the term “purified” refers to a Cannabinoid (a “purified Cannabinoid”) that is separated from all material that was present in the plant matter from which it was derived.

In one embodiment, the term “purified” refers to a Terpene (a “purified Terpene”) that is separated from other Cannabinoids that were present in the plant matter from which it was derived. In one embodiment, the term “purified” refers to a Terpene (a “purified Terpene”) that is separated from Terpenes that were present in the plant matter from which it was derived. In one embodiment, the term “purified” refers to a Terpene (a “purified Terpene”) that is separated from secondary compounds that were present in the plant matter from which it was derived. In one embodiment, the term “purified” refers to a Terpene (a “purified Terpene”) that is separated from all material that was present in the plant matter from which it was derived.

Carrier Oil

For purposes of this application, the term “Carrier Oil” is defined to include vegetable oils that are pressed from the fatty portions (seeds, nuts, kernels). Examples of Carrier Oils include, but are not limited to, the following: Acai palm oil, Almond oil, Apricot oil, Argan oil, Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil, Brazil nut oil, Buriti oil, Calendula oil, Camellia seed oil, Canola, Carapa oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Emu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil, Kukui nut oil, Linseed oil, Macadamia oil, Manketti nut oil, Marula oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil, Rice bran oil, Rosehip oil, Safflower, Sea Buckthorn oil, Safflower oil, Salicornia oil, Sesame seed oil, Solarium oil, Soybean oil, Strawberry seed oil, Sunflower seed oil, St John's Wort Oil, Sweet Almond oil, Tamanu oil, Tomato seed oil, Trauma oil, Vegetable oil, Vigna mungo oil, Walnut oil, Watermelon seed oil, Wheat germ oil, and similar or equivalent oils.

Essential Oils

For purposes of this application, the term “Essential Oil” is defined to include oils that are distilled from the aromatic leaves, bark, and roots of plants, and which have a concentrated aroma, such as is commonly known to those skilled in the art. Essential Oils are obtained only by steam distillation, hydro-distillation, or vacuum distillation of various aromatic parts of a botanical, including the leaves, roots, bark, steam, or flowers. Some of the main chemical constituents found in Essential Oils include Alcohols, Aldehydes, Esters, Ethers, Ketones, Phenols, and Terpenes. Examples of suitable Essential Oils include, but are not limited to, the following: Bergamot, Black Pepper, Blood Orange, Neroli Essential Oil, Peppermint Essential Oil, Spearmint Essential Oil, Lavender Essential Oil, Lemongrass Essential Oil. In some embodiments, the Cannabis Oil composition includes one or more added Essential Oils including but not limited to the following: Sweet Orange (Citrus sinensis spp), Peppermint (Mentha piperita spp), Lemon (Citrus limon spp), Lavender (Lavendula angustifolia spp) and Vanilla (Vanilla planifolia spp), Agarwood; Agarwood Attar; Arnica; Ahibero; Allspice; Almond, bitter; Amber Oil; Ambrette Seed; Amyris; Angelica Root; Angelica Seed; Aniseed; Anise; Anise (star); Armoise (Mugwort); Artemisia vestita; Asafoetida; Bakul; Balsam of Peru Oil; Balsam of Peru Resin; Balsamite; Baobab Oil; Basil, Sweet ct Linalool; Basil, Sweet ct Linalool—Organic; Basil, Sweet ct Methyl Chavicol—Organic; Bay; Beeswax; Bergamot; Birch; Boldo; Boronia; Black Cumin; Black Currant Bud; Black Pepper; Blue Lotus Attar; Broom; Buchu; Bupleurum (Bupleurum fruticosum); Buddha wood; Butter; Cabreuva; Cade; Cajuput; Calamus; Calendula; Camomile (or Chamomile); Camphor; Cananga; Cangerana; Cape Chamomile (Ericephalus punctulatus) S. Africa, Wild Harvest; Cape May; Caraway; Caraway; Cardamom; Carnation; Carrot Seed; Cascarilla; Cassia; Cassie; Catnip; Cedar (Cedrus) India; Cedarwood; Cedarwood, Atlas—Organic; Cedarwood, Himalayan; Cedarwood, Texas; Cedarwood, Virginia; Celery leaf, Celery Seed; Chamomile, Blue; Chamomile; Chamomile, Roman (Anthemis nobilis); Champa Attar (Michelia champaca) India; Champaca; Chaste tree; Cilantro; Cinnamon; Cinnamon Bark; Cistus; Cistus (Cistus ladaniferus) Corsica; Citronella; Clary Sage Absolute; Clary Sage, Bulgaria; Clary Sage, Russia; Clary Sage, USA; Clementine; Clove; Clove Bud; Cacao; Coconut Pulp; Coffee Bean Oil; Cognac, Green; Coleus; Combava (fruit or leaf); Copaiba; Coriander; Coriander Seed; Cucumber Hydrosol; Cumin; Cumin Seed; Cypress Leaf, Cypress, Blue; Davana; Dill; Elemi; Eucalyptus, Blue Gum; Eucalyptus, Blue Mallee; Eucalyptus, Lemon; Fennel (Foeniculum vulgare) Bulgaria; Fennel, Sweet; Fenugreek; Fern (sweet); Fleabane; Fir Needle; Fir, Balsam; Fir, Douglas; Fir, Silver; Fragonia; Frankincense, India; Frankincense, Somalia; Frankincense Frereana; Frankincense, Oman; Frankincense, Oman; Frankincense, Somalia; Galangal; Galbanum; Geranium; Geranium, Egypt; Geranium, Rose; Geranium, South Africa; Ghandi root; Ginger; Ginger Lily; Ginger, Fresh; Gingergrass (Cymbopogon martinii); Goldenrod; Grapefruit, Pink; Grapefruit, Ruby Red; Grapefruit, White; Hay; Helichrysum, Albania; Helichrysum, Croatia; Hina Attar, India; Hop; Hyssop Decumbens; Hyssop; Immortelle; Jasmine Absolute, Egypt; Jasmine Absolute, India; Jasmine Concrete; Jasmine; Jasmine Sambac; Jatamansi, (Nardostachs jatamansi) Juniper; Juniper Berry (Juniperus communis) or leaf, Kaffir Lime; Kava Kava; Labdanum; Larch needle; Laurel (Laurus nobilis) Corsica; Laurel Leaf, Lavandin, Grosso; Lavender—High Elevation; Lavender—Wild; Lavender Absolute; Lavender Hydrosol; Lavender, Bulgaria; Lavender, France; Lavender, Maillette; Leleshwa; Lemon; Lemon Tea Tree; Lemon verbena; Lemongrass; Lentisque (Pistacia lentiscus) Corsica; Lime; Lime Essence Oil; Lime, Distilled; Liquidambar (Styrax); Longoza; Lotus Absolute, Pink; Lotus Absolute, White; Lovage leaf; Lovage root; Magnolia flower; Mandarin; Mandarin, Green; Mandarin, Red; Mandarin, Yellow; Mango ginger; Marjoram; Manila oil; Melissa; Mint; Mint, Himalayan (Mentha arvensis); Mitti Attar; Motia Attar (Jasmine sambac) India; Mugwort; Mustard; Myrrh; Myrtle, Green; Myrtle (Myrtus Communis); Nagarmotha (Cypriol); Neem (Azadirachta indicd) India; Neroli; Niaouli; Nutmeg; Nut grass; Oakmoss Absolute; Oakwood; Opopanax, Sweet Myrrh (Commiphora guidotti); Orange, Blood; Orange, Sweet; Orange, Wild; Orange Blossom; Orange Essence Oil; Orange, Bitter Green; Orange, Bitter Red; Oregano; Orris Butter; Osmanthus Absolute; Palmarosa; Palmarosa, Nepal; Palmarosa, Sri Lanka; Palo Santo (Bursera graveolens); Palo Santo; Patchouli; Absolute; Patchouli, Dark; Patchouli, Light; Patchouli, Sri Lanka; Pennyroyal; Pepper, Black; Peppercorn, Pink; Peppermint, Chocolate; Peppermint, France; Petitgrain Absolute; Petitgrain Bigarade; Petitgrain sur Fleurs; Petitgrain, Mandarin; Pimento; Pine; Pinion Juniper Co-distillation, Colorado, Wild Harvest; Pinon Pine (Pinus edulis) Colorado, Wild Harvest; Pitta blend (Lavender, Rose Geranium, Ruh Khus); Plai; Pomegranate Seed; Rhododendron (Rhododendron anthopogon); Rhododendron Leaf, Rosalina; Rose; Rose Attar; Rose de Mai Absolute; Rose de Mai Concrete; Rose de Mai Organic Extract; Rose geranium; Rose Hip Seed; Rose Otto, Bulgaria; Rose Otto, Turkey; Rose Otto, White—Organic; Rose vetiver; Rosemary Antioxidant; Rosemary ct Cineole; Rosemary ct Verbenone; Rosewood; Rue; Ruh Khus (Vetiveria zizaniodes); Saffron Attar, India; Sage; Samphire (Cristhmum maritimum) Corsica; Sandalwood; Sandalwood, New Caledonia; Sandalwood, Australian—Premium; Sandalwood (Santalum spicatum), Australia; Sandalwood Oil, Royal Hawaiian (Santalum paniculatum); Sandalwood, Royal Hawaiian; Sassafras; Savitri Rose Perfume; Sea Buckthorn; Seaweed; Sierra Juniper (Juniperus occidentalis); Spearmint; Spearmint (Mentha Spicatd) Israel; Spikenard; Spikenard, Green; Spruce, Black; Spruce (Picea mariand) Canada; St. John's Wort (Hypericum perforatum) Bulgaria; Tagetes; Tamanu (Foraha) Oil; Tangelo; Tangerine; Tangerine Murcott; Tansy; Tansy, Blue; Tarragon; Tea Tree; Tea Tree (Leptospermum citratum), Lemon Scented; Tea Tree (Melaleuca alternifolid) South Africa; Thuja; Thyme; Thyme ct Linalool; Tobacco; Tonka Bean; Tuberose; Tulsi, Holy Basic Oil (Ocimum sanctum), Turmeric; Vanilla; Vanilla Bourbon; Verbena; Vetiver—Double Distilled; Vetiver, El Salvador; Vetiver, Haiti; Vetiver, Sri Lanka; Violet Leaf, White Fir (Abies concolor), White Lotus Attar; White Sage (Salvia apiana), Wild Carrot, Corsica; Wintergreen; Wintergreen; Yarrow; Yarrow, Blue; Ylang Ylang; and Yuzu.

Essential Oils can also provide antioxidant and preservative properties in the Cannabis Oil compositions. The identity and amount of the Essential Oil(s) added can depend in part on factors including the strain of cannabis that has been extracted and the desired organoleptic properties. In general, the amount of total Essential Oils added to a cannabis extract will range from about 0.01% (w/w) to about 10% (w/w) or more. The % (w/w) values indicated are based on the amount of Essential Oil added to the amount of total cannabis extract (including Vitamin E TPGS or additives other than the Essential Oil, if applicable). Lipophilic ingredients such as Cannabinoids, Terpenes, Vitamins/Antioxidants, Essential Oils, etc., have limitations such as poor stability and aqueous solubility, very low permeability that limits oral and topical delivery, leading to low bioavailability, absorption, permeation, and low potency. The goal is to overcome these limitations to exert maximum activity by improving physical and oxidative stability, creating shelf life stability, increasing oral and topical bioavailability, increasing permeability/absorption, and increasing the potency.

There is interplay between the lipids, surfactant, and excipients to produce stable o/o emulsion formulations. This novel preparation and production of stable o/o emulsions is dependent upon the appropriate selection and proper ratios between lipophilic ingredients (THC, CBD, Vitamins, Antioxidants, Terpenes, etc.), surfactant (TPGS) and excipients (Carrier Oils) to ensure chemical stability and to limit the risk of phase separation over storage time. These o/o emulsions complement traditional emulsions that utilize an aqueous phase and allows for the use of materials incompatible with water.

The most commonly studied emulsions are oil-in-water (o/w), water-in-oil (w/o), and double/multiple emulsions (e.g., o/w/o or w/o/w). In o/w emulsions, oil droplets are dispersed in water, whereas in w/o emulsions water droplets are dispersed in oil. In both systems, a low concentration of an appropriate surfactant (emulsifier/particle stabilizer) is commonly used to stabilize the interface. Although o/w and w/o emulsions have found widespread use across a number of fields, the presence of water in these systems limits the type of functionalities that can be used. Non-aqueous o/o emulsions are complementary to traditional o/w emulsions and attractive for a number of applications not compatible with water or aqueous systems. The past decade has seen a significant increase in both the design and application of o/o emulsions. This has been primarily driven by developments in understanding the mechanism of effective stabilization of o/o emulsion systems. Of note, o/o emulsions do not have to be strictly water free, but can be used to access a water/liquid environment.

Stabilization of the oil-water interface in emulsions can be realized using small molecules, block copolymers (BCPs), or particles. One of the most important features about the type of small molecule emulsifiers and the respective emulsions they stabilize is known as the hydrophilic-lipophilic balance (HLB), which is the balance between the size and strength of the two components of the emulsifier, i.e., hydrophilic and lipophilic components. HLB values have mainly been explored for small molecule surfactants, but have also found application in the case of amphiphilic macromolecules. For small molecules, emulsifiers with HLB values greater than 10 works better with o/w emulsions and HLB values lower than 10 work better with w/o emulsions, such that the HLB values of the emulsifier can be used to determine the type of emulsion formed. Carrier Oils (HLB range=6-11), Tocopheryl Polyethylene Glycol Succinate, TPGS, (HLB=13.2), amphiphilic small molecule emulsifiers such as sodium lauryl sulfate (HLB=40), sodium dodecylbenzenesulphonate (HLB=10.6), alkyl phenyl polyoxyethylene ether (HLB=14.5), and polyoxyethylene sorbitan monooleate (HLB=15), lead to the formation of o/w emulsions. In contrast, Span 80 (Sorbitan monooleate, HLB=4.9), Glyceryl monostearate (HLB=3.8) and Span 65 (Sorbitan tristearate, HLB=2.1) favor w/o emulsions. While small molecule surfactants, BCPs and Pickering particles are commonly used in the case of o/o emulsions.

When the o/o emulsion comes in contact with an aqueous solution within the mouth, stomach, and small intestines, nano or micro sized droplets (micelles) are formed and the absorption process begins. The small nature of the droplets increases the surface area available for lipophilic ingredients to be dissolved and absorbed, increasing the extent and rate in which the lipids can enter the circulatory system; increasing the bioavailability and the potency to the system. The unique combination of TPGS, excipients, and other lipophilic ingredients also creates a synergistic formulation to increase oral and topical absorption and bioavailability through several other mechanisms as well.

As previously mentioned, these formulations are used to create a variety of different products in liquid or solid form, that are ingestible, sublingual tinctures, beverages, soft gels, chewables, tablets, toothpaste, topical skin care products, or administered in any manner known in the art, and may contain liposomes, micelles, and or microspheres. The products are useful, for example, as a sleep aid, a treatment for anxiety, for pain relief, energy, gingivitis, and topical products for pain/muscle, skin regeneration, skin protection, skin healing, etc. While some examples of final products are described, this should be considered exemplary in nature, and not a limiting list, as those skilled in the art may adapt these processes to a wide variety of final products. For topical administration, this oil composition may be administered in a form that might include and is not limited to: an ointment, cream, lotion, gel, paste, solution, serum, etc., and may contain liposomes, micelles, and or microspheres to address issues with muscle pain, inflammation, bruising, skin regeneration, skin protection, skin healing, acne, psoriasis, gingivitis, etc.

A wide variety of different ratios and manufacturing techniques were tested to find the optimal ratios of Vitamin E TPGS with Cannabinoids, Terpenes, Carrier Oil(s) and Essential Oil(s) to create stable liquid o/o emulsions. The optimal ratios were determined to create stable liquid emulsions. Various formulations are discussed in the following examples; however, these are illustrative of the invention, and should not be considered limiting, but the invention includes equivalent formulations consistent with the teachings of the current disclosure.

Heat and Atmospheric Pressure Manufacturing Process

In some embodiments, at least one heat-safe container may be used in this process for holding the ingredients described herein. The container(s) is heated by a heat source, in this case a magnetic hot plate, to a temperature between 30-100 degrees Celsius (86-212 degrees Fahrenheit), (optimal temperature is between 104-176F). In various embodiments, the ingredients can be heated and mixed with sonication, ultrasonication, stirring, mixing, homogenization or external energy. In this embodiment, mixing of ingredients is performed at between 200-1000 rotations per minute (optimally between 430-470 RPM).

Method One:

At a first step of the method for forming an emulsion, Vitamin E TPGS, a Cannabinoid, and the Carrier Oils are each measured into a separate container and then placed on a magnetic hot plate stirrer 40°−80° C. (104°−176° F.), while mixing, for approximately five (5) minutes to ensure homogeneity of each liquid. In some embodiments, Essential Oil(s) or CoQ₁₀ is also measured and heated in the same manner.

At a next step of the method, the heated Carrier Oil(s) is slowly added to the heated Cannabinoid while stirring to form a first composition. If CBD Isolate is used, stir for approximately fifteen (15) minutes. If THC Distillate is used, stir for approximately five (5) minutes. If CoQ₁₀ or Essential Oil(s) are used, the heated Carrier Oil(s) are first slowly added to the heated CoQ₁₀ or Essential Oil(s) and stirred for ten (10) minutes before adding to the heated Cannabinoid, while mixing.

Next, the first composition is slowly added to the heated Vitamin E TPGS while stirring to form a second composition, which is stirred for approximately fifteen (15) minutes while maintaining a temperature of approximately 70° C. (158° F.).

Finally, the second composition is mixed at 30-100 degrees Celsius (86-212 degrees Fahrenheit) for 10-20 minutes. The heat may then be turned off, wherein the second composition is mixed until it is at room temperature and the emulsion is formed. The contents are stirred until all the ingredients are homogenous and the temperature is even throughout the entire emulsion. Additional ingredients, as described below, may also be added to this composition, either before or after heating. Some of these various alternative methods are discussed in greater detail below.

Example ratios for Vitamin E TPGS, Cannabinoid, and Carrier Oil(s) using this method are as follows:

Using CBD Isolate: (1:1.0) (1:1.7) (1:2.0) (1:2.3) (1:2.7) (1:3.0) (1:3.5) (1:4.0) (1:4.6) (1:5.0) (1:6.0)

Using THC Distillate: (1:1.0) (1:2.0) (1:2.3) (1:2.7) (1:3.0) (1:3.5) (1:4.0) (1:4.6) (1:5.0)

Using Essential Oil(s) or CoQ₁₀: (1:2.3) (1:3.0) (1:3.5) (1:4.0) (1:5.0) (1:6.0)

Method Two:

At a first step of the method for forming an emulsion, Vitamin E TPGS, a Cannabinoid, and the Carrier Oils are each measured into the same container and then placed on a magnetic hot plate stirrer 40°−80° C. (104°−176° F.), while mixing, for approximately five (5) minutes to ensure homogeneity of each liquid. In some embodiments, Essential Oil(s) or CoQ₁₀ is also measured and heated in the same manner. The ingredients can be heated and mixed with sonication, ultrasonication, stirring, mixing, homogenization or external energy.

Finally, the second composition is mixed at 30-100 degrees Celsius (86-212 degrees Fahrenheit) for 10-20 minutes. The heat may then be turned off, wherein the second composition is mixed until it is at room temperature and the emulsion is formed. The contents are stirred until all the ingredients are homogenous and the temperature is even throughout the entire emulsion.

Example ratios for Vitamin E TPGS, Cannabinoid, and Carrier Oil(s) using this method are as follows: For CBD Isolate: (1:1.0) (1:1.3) (1:1.7) (1:2.0) (1:2.3) (1:2.7) (1:3.0) (1:3.5) (1:4.0) (1:4.6) (1:5.0) (1:6.0); For THC Distillate: (1:1.0) (1:2.0) (1:2.3) (1:2.7) (1:3.0) (1:3.5) (1:4.0) (1:4.6) (1:5.0); and Using Essential Oil(s) or CoQ₁₀: (1:2.3) (1:3.0) (1:3.5) (1:4.0) (1:5.0) (1:6.0).

A wide variety of different Cannabinoid emulsions have been prepared, using a combination of different Cannabinoids and or Terpenes with a combination of Carrier Oils or just one type of Carrier Oil and Vitamin E TPGS. These emulsions may further include Essential Oils, and Coenzyme CoQ₁₀. Long Chain Triglycerides (LCT) and/or fatty acids found in Carrier Oils, act as stabilizers. Thus, we prefer to use Carrier Oils that are high in LCT and Linoleic Acid. Carrier Oils, also known as base oil or vegetable oil, have different viscosities and are comprised of a wide variety of different fatty acids and different ratios of fatty acids. The most common Saturated fatty acids (SFA) include: Stearic acid, Palmitic acid, Myristic acid, Lauric acid, Capric acid, Caprylic acid, Caproic acid. Most common Monounsaturated fatty acids (MUFA) include: Oleic acid, Elaidic acid, Palmitoleic acid, Vacentic acid. Most common Polyunsaturated fatty acids (PUFA) include: Omega-3 fatty acids (Alpha-linolenic acid), Polyunsaturated Omega-6 (Linoleic acid), Omega-7 (Palmitoleic acid) fatty acids. The formulated emulsions, which may be stored in sealed containers for 1½-2 years, are still stable, and none of the oils have separated.

Mixing just Vitamin E TPGS without a Carrier Oil(s) with other types of oil based (lipophilic) ingredient(s) does not create a stable liquid emulsion if the ratio of Vitamin E TPGS to lipophilic ingredient(s) is greater than (1:1.3). A ratio of (1:1 or 1:2) will create a stable emulsion that is extremely thick at room temperature. This emulsion is too dense and thick; it cannot be used with manufacturing equipment to produce consumable products such as soft gel capsules. If a Carrier Oil(s) is used with Vitamin E TPGS and a lipophilic ingredient(s) with the correct formula/ratio, a stable oil/oil (o/o) emulsion can be created at ratios greater than (1:1.0). This could include Essential Oil(s), Terpene(s), lipophilic Vitamin(s) and antioxidant(s), or Long-Chain Triglycerides (LCT) and Medium-Chain Triglycerides (MCT) with one or more of the following poorly soluble lipophilic compounds/ingredients: Cannabinoids, Terpenes, Lipophilic Vitamins/Antioxidants, (which improve oral and topical absorption/permeability and bioavailability). Extensive testing demonstrated that various ratios of LCT and/or MCT, along with Carrier Oil(s) and other lipophilic ingredients will create a stable o/o lipophilic emulsion.

Once formulated, you can apply directly onto your skin or hair, or swallow, and it will be absorbed. You can place under your tongue for faster absorption.

After oral administration, the o/o emulsion consisting of Carrier Oil(s), Vitamin E TPGS, Cannabinoids, Terpenes, Lipophilic Vitamins/Antioxidants, etc., enhances permeation across a variety of cellular membranes, including those in the mouth and gut. The o/o emulsion, mix with saliva in the mouth, gastrointestinal fluids where spontaneous emulsification occurs and produces mixed micelles. These mixed micelles are absorbed by the enterocytes where chylomicrons formation occurs. Chylomicrons along with the Cannabinoids are transported into the lymphatic vessel, thus bypassing the direct transport to the liver, thereby increasing absorption and increasing onset times.

Particular Manufacturing Embodiments:

Manufacturing Process Number 1A:

Each ingredient was measured in a separate beaker, then heated separately and then slowly added together. Different ratios of Vitamin E TPGS to CBD Isolate were tested at these different ratios: (1:1.0) (1:1.3) (1:1.7) (1:2.0) (1:2.3) (1:2.7) (1:3.0) (1:3.5) (1:4.0) (1:4.6) (1:5.0) (1:6.0) ratios. Vitamin E TPGS was measured, added to a beaker, placed on a magnetic hot plate stirrer, heated between 40°−80° C. (104°−176° F.) and stirred for five (5) minutes to ensure homogeneity of the liquid. CBD Isolate was measured, added to a beaker, placed on a magnetic hot plate stirrer, heated between 40°-80° C. (104°-176° F.) and stirred for five (5) minutes to ensure homogeneity of the liquid. The heated Vitamin E TPGS was stirred at 450 rpm. While the heated Vitamin E TPGS was being stirred, the heated CBD Isolate was then added slowly to the beaker containing the heated Vitamin E TPGS. This melted combination of Vitamin E TPGS and CBD Isolate (mixture) was stirred at 450 rpm for fifteen (15) minutes, while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The liquid mixture/emulsion was allowed to sit at room temperature in a dark area and observed everyday for stability. Within one (1) week, emulsion ratios (1:1.0) and (1:1.3) did not separate. After ten (10) months, these emulsion ratios did not separate; they were stable. Emulsion ratios (1:1.7) (1:2.0) (1:2.3) (1:2.7) (1:3.0) (1:3.5) (1:4.0) (1:4.6) (1:5.0) (1:6.0) separated or crystallized within days or weeks, they are not stable.

| | | Vitamin E TPGS | CBD Isolate | |
|---|---|---|---|---|
| Sample | Ratio | (g) wt | (g) wt | Stability |
| 1 | 1:1.0 | 3.75 | 3.75 | stable |
| 2 | 1:1.3 | 3.50 | 4.55 | stable |
| 3 | 1:1.7 | 3.00 | 5.10 | unstable |
| 4 | 1:2.0 | 2.50 | 5.00 | unstable |
| 5 | 1:2.3 | 2.50 | 5.75 | unstable |
| 6 | 1:2.7 | 2.25 | 6.07 | unstable |
| 7 | 1:3.0 | 2.00 | 6.00 | unstable |
| 8 | 1:3.5 | 2.00 | 7.00 | unstable |
| 9 | 1:4.0 | 1.80 | 7.20 | unstable |
| 10 | 1:4.6 | 1.50 | 6.90 | unstable |
| 11 | 1:5.0 | 1.50 | 7.50 | unstable |
| 12 | 1:6.0 | 2.00 | 12.00 | unstable |

The ingredients can be heated and mixed with sonication, ultrasonication, stirring, mixing, homogenization or external energy. The two stable liquid emulsion ratios (1:1.0) and (1:1.3) have a very high viscosity, which makes these ratios to thick for manufacturing of soft gels or capsules and makes it nearly impossible to manufacture other types of consumable products such as soft gels, tablets, edibles, gummies, cookies, candy, beverages, skin care products, liquids, sprays, creams, topical applications, etc.

1) Emulsion Stability Test Using Vitamin E TPGS with CBD Isolate.

Manufacturing Process Number 1B:

All of the lipophilic ingredients were heated together. Vitamin E TPGS was combined with CBD Isolate and the liquid emulsions were tested for stability at these different ratios: (1:1.0) (1:1.3) (1:1.7) (1:2.0) (1:2.3) (1:2.7) (1:3.0) (1:3.5) (1:4.0) (1:4.6) (1:5.0) (1:6.0). CBD Isolate was measured, added to a beaker. Vitamin E TPGS was measured and added to the same beaker containing the CBD Isolate. The beaker with CBD Isolate and Vitamin E TPGS was placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The liquid mixture/emulsions were allowed to sit at room temperature in a dark area and observed everyday for stability. Within one (1) week, emulsion ratios (1:1) and (1:1.3) did not separate. After ten (10) months, these emulsion ratios did not separate; they were stable. Emulsion ratios (1:1.7) (1:2.0) (1:2.3) (1:2.7) (1:3.0) (1:3.5) (1:4.0) (1:4.6) (1:5.0) (1:6.0) separated or crystallized within days or weeks, they are not stable.

| | | Vitamin E TPGS | CBD Isolate | |
|---|---|---|---|---|
| Sample | Ratio | (g) wt | (g) wt | Stability |
| 1 | 1:1.0 | 3.75 | 3.75 | stable |
| 2 | 1:1.3 | 3.50 | 4.55 | stable |
| 3 | 1:1.7 | 3.00 | 5.10 | unstable |
| 4 | 1:2.0 | 2.50 | 5.00 | unstable |
| 5 | 1:2.3 | 2.50 | 5.75 | unstable |
| 6 | 1:2.7 | 2.25 | 6.07 | unstable |
| 7 | 1:3.0 | 2.00 | 6.00 | unstable |
| 8 | 1:3.5 | 2.00 | 7.00 | unstable |
| 9 | 1:4.0 | 1.80 | 7.20 | unstable |
| 10 | 1:4.6 | 1.50 | 6.90 | unstable |
| 11 | 1:5.0 | 1.50 | 7.50 | unstable |
| 12 | 1:6.0 | 2.00 | 12.00 | unstable |

The ingredients can be heated and mixed with sonication, ultrasoni cation, stirring, mixing, homogenization or external energy. The two emulsion stable ratios (1:1.0) and (1:1.3) have a very high viscosity, which makes these ratios too thick for manufacturing of soft gels or capsules and makes it almost impossible to manufacture other types of consumable products such as: hard or soft gelatin capsules, hard gummies, cookies, candy, beverages, skin care products, etc.

2) Emulsion Stability Test Using Carrier Oil(s), Vitamin E TPGS, and CBD Isolate

Combine Vitamin E TPGS, CBD Isolate, and Carrier Oil(s) Together at Different Ratios and Test the Emulsions for Stability, Two (2) Different Manufacturing Processes were Performed.

Manufacturing Process Number 2A:

Ingredients were heated separately and then slowly added together. Vitamin E TPGS was combined with different ratios of CBD Isolate and Carrier Oil(s) the liquid emulsions were tested for stability at these ratios: (1:1.0) (1:1.3) (1:1.7) (1:2.0) (1:2.3) (1:2.7) (1:3.0) (1:3.5) (1:4.0) (1:4.6) (1:5.0) (1:6.0). Vitamin E TPGS was measured, added to a beaker, placed on a magnetic hot plate stirrer and heated between 40°−80° C. (104°−176° F.) and stirred for five (5) minutes. CBD Isolate was measured, added to a beaker, placed on a magnetic hot plate stirrer and heated between 40°-80° C. (104°-176° F.) and stirred for five (5) minutes to ensure homogeneity of the liquid. The Carrier Oil(s) was measured, added to a beaker, placed on a magnetic hot plate stirrer and heated between 40°-80° C. (104°-176° F.) and stirred for five (5) minutes to ensure homogeneity of the liquid. The heated Vitamin E TPGS was stirred at 450 rpm. The heated Carrie Oil(s) was slowly added to the heated CBD Isolate and stirred for fifteen (15) minutes to ensure homogeneity of the liquid. The heated mixture of CBD Isolate and Carrier Oil(s) was then added slowly to the beaker containing the heated Vitamin E TPGS. This melted combination of Vitamin E TPGS, CBD Isolate and Carrier Oil(s) (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, emulsion ratios (1:1.0) (1:1.3) (1:1.7) (1:2.0) (1:2.3) (1:2.7) (1:3.0) (1:3.5) (1:4.0) (1:4.6) (1:5.0) (1:6.0) did not separate; they were stable. After ten (10) months, these emulsion ratios did not separate, they are stable.

| | | Vitamin E TPGS | CBD Isolate | Carrier Oil | |
|---|---|---|---|---|---|
| Sample | Ratio | (g) wt | (g) wt | (g) wt | Stability |
| 1 | 1:1.0 | 4.00 | 2.00 | 2.00 | stable |
| 2 | 1:1.3 | 3.00 | 1.95 | 1.95 | stable |
| 3 | 1:1.7 | 3.00 | 2.55 | 2.55 | stable |
| 4 | 1:2.0 | 3.00 | 3.00 | 3.00 | stable |
| 5 | 1:2.3 | 2.50 | 2.88 | 2.88 | stable |
| 6 | 1:2.7 | 2.50 | 3.37 | 3.37 | stable |
| 7 | 1:3.0 | 2.50 | 3.75 | 3.75 | stable |
| 8 | 1:3.5 | 2.00 | 3.50 | 3.50 | stable |
| 9 | 1:4.0 | 2.00 | 4.00 | 4.00 | stable |
| 10 | 1:4.6 | 2.00 | 4.60 | 4.60 | stable |
| 11 | 1:5.0 | 2.00 | 5.00 | 5.00 | stable |
| 12 | 1:6.0 | 2.00 | 6.00 | 6.00 | stable |

The ingredients can be heated and mixed with sonication, ultrasonication, stirring, mixing, homogenization or external energy.

Manufacturing Process Number 2B:

All the ingredients were heated together. Vitamin E TPGS was combined with different ratios of CBD Isolate, and Carrier Oil(s), the liquid emulsions were tested for stability at these ratios: (1:1.0) (1:1.3) (1:1.7) (1:2.0) (1:2.3) (1:2.7) (1:3.0) (1:3.5) (1:4.0) (1:4.6) (1:5.0) (1:6.0). CBD Isolate was measured, added to a beaker. Vitamin E TPGS was measured and added to the same beaker containing the CBD Isolate. Carrier Oil(s) was measured and added to the same beaker containing the Vitamin E TPGS and CBD Isolate. The beaker with CBD Isolate, Vitamin E TPGS, and Carrier Oil(s) was then placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, emulsion ratios (1:1.0) (1:1.3) (1:1.7) (1:2.0) (1:2.3) (1:2.7) (1:3.0) (1:3.5) (1:4.0) (1:4.6) (1:5.0) (1:6.0) did not separate; they were stable. After ten (10) months, these emulsion ratios did not separate, they are stable.

| | | Vitamin E TPGS | CBD Isolate | Carrier Oil | |
|---|---|---|---|---|---|
| Sample | Ratio | (g) wt | (g) wt | (g) wt | Stability |
| 1 | 1:1.0 | 4.00 | 2.00 | 2.00 | stable |
| 2 | 1:1.3 | 3.00 | 1.95 | 1.95 | stable |
| 3 | 1:1.7 | 3.00 | 2.55 | 2.55 | stable |
| 4 | 1:2.0 | 3.00 | 3.00 | 3.00 | stable |
| 5 | 1:2.3 | 2.50 | 2.88 | 2.88 | stable |
| 6 | 1:2.7 | 2.50 | 3.37 | 3.37 | stable |
| 7 | 1:3.0 | 2.50 | 3.75 | 3.75 | stable |
| 8 | 1:3.5 | 2.00 | 3.50 | 3.50 | stable |
| 9 | 1:4.0 | 2.00 | 4.00 | 4.00 | stable |
| 10 | 1:4.6 | 2.00 | 4.60 | 4.60 | stable |
| 11 | 1:5.0 | 2.00 | 5.00 | 5.00 | stable |
| 12 | 1:6.0 | 2.00 | 6.00 | 6.00 | stable |

The ingredients can be heated and mixed with sonication, ultrasoni cation, stirring, mixing, homogenization or external energy. It has been shown through hundreds of tests—Carrier Oil(s) need to be used within certain ratios with other lipophilic ingredients to create a stable o/o lipophilic emulsion. There are ratios within these ratios to create stable o/o emulsions. For example, in Sample 9 above, the ratio of CBD Isolate to the Carrier Oil is 50/50. If I change this ratio of CBD Isolate to the Carrier Oil to 80/20 or 35/65 and keep the ratio of Vitamin E TPGS to all the oils at 1:4.0 the o/o emulsion most likely won't be stable.

3) Combine Vitamin E TPGS and THC Distillate Together at Different Ratios and Test for Emulsion Stability; Two (2) Different Manufacturing Processes were Performed.

Manufacturing Process Number 3A

Ingredients were heated separately and then slowly added together. Vitamin E TPGS was combined with different ratios of THC Distillate and the liquid emulsions were tested for stability at these ratios: (1:1) (1:2.0) (1:2.3) (1:2.7) (1:3.0) (1:3.5) (1:4.0) (1:4.6) (1:5.0). Vitamin E Vitamin E TPGS was measured, added to a beaker, placed on a magnetic hot plate stirrer and heated between 40°−80° C. (104°−176° F.) and stirred for five (5) minutes to ensure homogeneity of the liquid. The THC Distillate was measured, added to a beaker, placed on a magnetic hot plate stirrer and heated between 40°-80° C. (104°-176° F.) and stirred for five (5) minutes to ensure homogeneity of the liquid. The heated Vitamin E TPGS was stirred at 450 rpm. While the heated Vitamin E TPGS was being stirred, the heated THC Distillate was then added slowly to the beaker containing the heated Vitamin E TPGS. This melted combination of Vitamin E TPGS and THC Distillate (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within days or weeks these liquid emulsion ratios (1:1.0) (1:2.0) (1:2.3) (1:2.7) (1:3.0) (1:3.5) (1:4.0) (1:4.6) (1:5.0) separated; they are not stable.

| | | Vitamin E TPGS | THC Distillate | |
|---|---|---|---|---|
| Sample | Ratio | (g) wt | (g) wt | Stability |
| 1 | 1:1.0 | 3.75 | 3.75 | unstable |
| 2 | 1:2.0 | 2.50 | 5.00 | unstable |
| 3 | 1:2.3 | 2.25 | 5.75 | unstable |
| 4 | 1:2.7 | 2.25 | 6.08 | unstable |
| 5 | 1:3.0 | 2.00 | 6.00 | unstable |
| 6 | 1:3.5 | 2.00 | 7.00 | unstable |
| 7 | 1:4.0 | 1.75 | 7.00 | unstable |
| 8 | 1:4.6 | 1.50 | 6.90 | unstable |
| 9 | 1:5.0 | 1.50 | 7.50 | unstable |

The ingredients can be heated and mixed with sonication, ultrasonication, stirring, mixing, homogenization or external energy.

Manufacturing Process Number 3B:

Ingredients were heated together. Vitamin E TPGS was combined with THC Distillate at different ratios and the liquid emulsions were tested for stability at these ratios: (1:1) (1:2.0) (1:2.3) (1:2.7) (1:3.0) (1:3.5) (1:4.0) (1:4.6) (1:5.0). THC Distillate was measured, added to a beaker. Vitamin E TPGS was measured and added to the same beaker containing the THC Distillate. The beaker with THC Distillate and Vitamin E TPGS was placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Emulsion ratios (1:1.0) (1:2.0) (1:2.3) (1:2.7) (1:3.0) (1:3.5) (1:4.0) (1:4.6) (1:5.0) separated within days or weeks, they are not stable.

| | | Vitamin E TPGS | THC Distillate | |
|---|---|---|---|---|
| Sample | Ratio | (g) wt | (g) wt | Stability |
| 1 | 1:1.0 | 3.75 | 3.75 | unstable |
| 2 | 1:2.0 | 2.50 | 5.00 | unstable |
| 3 | 1:2.3 | 2.25 | 5.75 | unstable |
| 4 | 1:2.7 | 2.25 | 6.08 | unstable |
| 5 | 1:3.0 | 2.00 | 6.00 | unstable |
| 6 | 1:3.5 | 2.00 | 7.00 | unstable |
| 7 | 1:4.0 | 1.75 | 7.00 | unstable |
| 8 | 1:4.6 | 1.50 | 6.90 | unstable |
| 9 | 1:5.0 | 1.50 | 7.50 | unstable |

The ingredients can be heated and mixed with sonication, ultrasonication, stirring, mixing, homogenization or external energy.

4) Combine Vitamin E TPGS, THC Distillate, and Carrier Oil(s) at Different Ratios and Tested for Emulsion Stability, Two (2) Different Manufacturing Processes were Performed.

Manufacturing Process Number 4A:

Ingredients were heated separately and then slowly added together. Vitamin E TPGS was combined with different ratios of THC Distillate and Carrier Oil(s), and then the liquid emulsions were tested for stability at these ratios: (1:1.0) (1:2.0) (1:2.3) (1:2.7) (1:3.0) (1:3.5) (1:4.0) (1:4.6) (1:5.0). Vitamin E TPGS was measured, added to a beaker, placed on a magnetic hot plate stirrer and heated between 40°−80° C. (104°−176° F.) and stirred for five (5) minutes to ensure homogeneity of the liquid. The THC Distillate was measured, added to a beaker, placed on a magnetic hot plate stirrer and heated between 40°-80° C. (104°-176° F.) and stirred for five (5) minutes to ensure homogeneity of the liquid. Carrier Oil(s) was measured, added to a beaker, placed on a magnetic hot plate stirrer and heated between 40°-80° C. (104°-176°) and stirred for five (5) minutes to ensure homogeneity of the liquid. The heated Carrier Oil(s) were added to the hot THC Distillate and stirred for five (5) minutes to ensure homogeneity of the liquid. The heated Vitamin E TPGS was stirred at 450 rpm. While the heated Vitamin E TPGS was being stirred, the heated THC Distillate and heated Carrier Oil(s) was then added slowly to the beaker containing the heated Vitamin E TPGS. The mix of Vitamin E TPGS, THC Distillate, and Carrie Oil(s) (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixtures/emulsions were allowed to sit at room temperature in a dark area and observed everyday for stability. Within one (1) week, emulsion ratios (1:1.0) (1:2.0) (1:2.3) (1:2.7) (1:3.0) (1:3.5) (1:4.0) (1:4.6) (1:5.0) did not separate. After ten (10) months, these emulsion ratios did not separate; they are stable.

| | | Vitamin E TPGS | THC Distillate | Carrier Oil | |
|---|---|---|---|---|---|
| Sample | Ratio | (g) wt | (g) wt | (g) wt | Stability |
| 1 | 1:1.0 | 3.75 | 1.87 | 1.87 | stable |
| 2 | 1:2.0 | 2.50 | 2.50 | 2.50 | stable |
| 3 | 1:2.3 | 2.25 | 2.87 | 2.87 | stable |
| 4 | 1:2.7 | 2.25 | 3.04 | 3.04 | stable |
| 5 | 1:3.0 | 2.00 | 3.00 | 3.00 | stable |
| 6 | 1:3.5 | 2.00 | 3.50 | 3.50 | stable |
| 7 | 1:4.0 | 1.75 | 3.50 | 3.50 | stable |
| 8 | 1:4.6 | 1.50 | 3.45 | 3.45 | stable |
| 9 | 1:5.0 | 1.50 | 3.75 | 3.75 | stable |

The ingredients can be heated and mixed with sonication, ultrasonication, stirring, mixing, homogenization or external energy.

Manufacturing Process Number 4B:

Ingredients were heated together. Vitamin E TPGS was combined with THC Distillate and Carrier Oil(s) at different ratios and then the liquid emulsions were tested for stability at these ratios: (1:1.0) (1:2.0) (1:2.3) (1:2.7) (1:3.0) (1:3.5) (1:4.0) (1:4.6) (1:5.0). THC Distillate was measured, added to a beaker. Carrier Oil(s) was measured and added to the same beaker containing the THC Distillate. Vitamin E TPGS was measured and added to the same beaker containing the THC Distillate, and Carrier Oil(s). The beaker with THC Distillate, Vitamin E TPGS, and Carrier Oil(s) was placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixtures/emulsions were allowed to sit at room temperature in a dark area and observed everyday for stability. Within one (1) week, emulsion ratios (1:1.0) (1:2.0) (1:2.3) (1:2.7) (1:3.0) (1:3.5) (1:4.0) (1:4.6) (1:5.0) remained stable. After ten (10) months, these emulsion ratios did not separate, they are stable.

| | | Vitamin E TPGS | THC Distillate | Carrier Oil | |
|---|---|---|---|---|---|
| Sample | Ratio | (g) wt | (g) wt | (g) wt | Stability |
| 1 | 1:1.0 | 3.75 | 1.87 | 1.87 | stable |
| 2 | 1:2.0 | 2.50 | 2.50 | 2.50 | stable |
| 3 | 1:2.3 | 2.25 | 2.87 | 2.87 | stable |
| 4 | 1:2.7 | 2.25 | 3.04 | 3.04 | stable |
| 5 | 1:3.0 | 2.00 | 3.00 | 3.00 | stable |
| 6 | 1:3.5 | 2.00 | 3.50 | 3.50 | stable |
| 7 | 1:4.0 | 1.75 | 3.50 | 3.50 | stable |
| 8 | 1:4.6 | 1.50 | 3.45 | 3.45 | stable |
| 9 | 1:5.0 | 1.50 | 3.75 | 3.75 | stable |

The ingredients can be heated and mixed with sonication, ultrasonication, stirring, mixing, homogenization or external energy.

5) Test the Emulsions for Stability without Carrier Oil(s). Only Vitamin E TPGS, CBD Isolate, and Essential Oil(s) were Combined. Two (2) Different Manufacturing Processes were Performed.

Manufacturing Process Number 5A:

Ingredients were heated separately and then slowly added together. Vitamin E TPGS was combined with different ratios of CBD Isolate, and Essential Oil(s), then the liquid emulsions were tested for stability at these ratios: (1:2.3) (1:3.0) (1:3.5) (1:4.0) (1:5.0) (1:6.0) Vitamin E TPGS was measured, added to a beaker, placed on a magnetic hot plate stirrer and heated between 40°−80° C. (104°−176° F.). CBD Isolate was measured, added to a beaker, placed on a magnetic hot plate stirrer and heated between 40°-80° C. (104°-176° F.). The Essential Oil(s) were measured, added to a beaker, placed on a magnetic hot plate stirrer and heated between 40°-80° C. (104°-176° F.). The heated Vitamin E TPGS was stirred at 450 rpm to ensure homogeneity of the liquid. The heated Essential Oil(s) were slowly added to the heated CBD Isolate and stirred for fifteen (15) minutes. The heated mixture of CBD Isolate and Essential Oil(s) were then added slowly added to the beaker containing the heated Vitamin E TPGS.

This melted combination of Vitamin E TPGS, CBD Isolate, and Essential Oil(s) (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixtures/emulsions were allowed to sit at room temperature in a dark area and observed everyday for stability. Emulsion ratios (1:2.3) (1:3.0) (1:3.5) (1:4.0) (1:5.0) (1:6.0) separated within days or weeks, they are not stable.

| | | | | Essential | |
|---|---|---|---|---|---|
| | | Vitamin E TPGS | CBD Isolate | Oils | |
| Sample | Ratio | (g) wt | (g) wt | (g) wt | Stability |
| 1 | 1:2.3 | 3.00 | 3.45 | 3.45 | unstable |
| 2 | 1:2.7 | 3.00 | 4.05 | 4.05 | unstable |
| 3 | 1:3.0 | 3.00 | 4.50 | 4.50 | unstable |
| 4 | 1:3.5 | 3.00 | 5.25 | 5.25 | unstable |
| 5 | 1:4.0 | 2.00 | 4.00 | 4.00 | unstable |
| 6 | 1:5.0 | 2.00 | 5.00 | 5.00 | unstable |
| 7 | 1:6.0 | 2.00 | 6.00 | 6.00 | unstable |

The ingredients can be heated and mixed with sonication, ultrasonication, stirring, mixing, homogenization or external energy.

Manufacturing Process Number 5B):

All the ingredients were heated together. Vitamin E TPGS was combined with different ratios of CBD Isolate, and Essential Oil(s), and then the liquid emulsions were tested for stability at these ratios: (1:2.3) (1:3.0) (1:3.5) (1:4.0) (1:4.6) (1:5.0) (1:6.0). CBD Isolate was measured, added to a beaker. Vitamin E TPGS was measured and added to the same beaker containing the CBD Isolate. Essential Oil(s) were measured and then added to the same beaker containing the Vitamin E TPGS, and CBD Isolate. The beaker with CBD Isolate, Vitamin E TPGS, and Essential Oil(s) were then placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Emulsion ratios (1:2.3) (1:3.0) (1:3.5) (1:4.0) (1:5.0) (1:6.0) separated within days or weeks, they are not stable.

| | | | | Essential | |
|---|---|---|---|---|---|
| | | Vitamin E TPGS | CBD Isolate | Oils | |
| Sample | Ratio | (g) wt | (g) wt | (g) wt | Stability |
| 1 | 1:2.3 | 3.00 | 3.45 | 3.45 | unstable |
| 2 | 1:2.7 | 3.00 | 4.05 | 4.05 | unstable |
| 3 | 1:3.0 | 3.00 | 4.50 | 4.50 | unstable |
| 4 | 1:3.5 | 3.00 | 5.25 | 5.25 | unstable |
| 5 | 1:4.0 | 2.00 | 4.00 | 4.00 | unstable |
| 6 | 1:5.0 | 2.00 | 5.00 | 5.00 | unstable |
| 7 | 1:6.0 | 2.00 | 6.00 | 6.00 | unstable |

The ingredients can be heated and mixed with sonication, ultrasonication, stirring, mixing, homogenization or external energy.

6) Test Emulsion Stability Using Carrier Oil(s).

Vitamin E TPGS, CBD Isolate, Essential Oil(s), and Carrier Oil(s), and were Combined.

Two (2) different manufacturing processes were performed.

Manufacturing Process Number 6A: Ingredients were heated separately and then slowly added together. Vitamin E TPGS was combined with different ratios of CBD Isolate, Carrier Oil(s), and Essential Oils, then the liquid emulsion was tested for stability at these ratios: (1:2.3) (1:3.0) (1:3.5) (1:4.0) (1:5.0) (1:6.0). Vitamin E TPGS was measured, added to a beaker, placed on a magnetic hot plate stirrer and heated between 40°−80° C. (104°−176° F.). CBD Isolate was measured, added to a beaker, placed on a magnetic hot plate stirrer and heated between 40°-80° C. (104°-176° F.). The Carrier(s) Oil was measured, added to a beaker, placed on a magnetic hot plate stirrer and heated between 40°-80° C. (104°-176° F.). The Essential Oils were measured, added to a beaker, placed on a magnetic hot plate stirrer and heated between 40°-80° C. (104°-176° F.). The heated Vitamin E TPGS was stirred at 450 rpm for fifteen (15) minutes to ensure homogeneity of the liquid. The heated Carrier Oil(s) were slowly added to the heated Essential Oils and stirred for ten (10) minutes to ensure homogeneity of the liquid. The heated Carrier Oil(s) and Essential Oil(s) were slowly added to the heated CBD Isolate and stirred for fifteen (15) minutes to ensure homogeneity of the liquid. The heated mixture of CBD Isolate, Carrier Oil(s), and Essential Oil(s) were then added slowly to the beaker containing the heated Vitamin E TPGS. This melted combination of Vitamin E TPGS, CBD Isolate, Carrier Oil(s), and Essential Oil(s) (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, emulsion ratios (1:2.3) (1:3.0) (1:3.5) (1:4.0) (1:5.0) (1:6.0) did not separate, they are stable. After ten (10) months, these emulsion ratios did not separate, they were stable.

| | | Vitamin E | CBD | Essential | Carrier | |
|---|---|---|---|---|---|---|
| | | TPGS | Isolate | Oils | Oil | |
| Sample | Ratio | (g) wt | (g) wt | (g) wt | (g) wt | Stability |
| 1 | 1:2.3 | 3.00 | 3.45 | 1.73 | 1.73 | stable |
| 2 | 1:2.7 | 3.00 | 4.06 | 2.03 | 2.03 | stable |
| 3 | 1:3.0 | 3.00 | 4.50 | 2.25 | 2.25 | stable |
| 4 | 1:3.5 | 3.00 | 5.25 | 2.63 | 2.63 | stable |
| 5 | 1:4.0 | 2.00 | 4.00 | 2.00 | 2.00 | stable |
| 6 | 1:5.0 | 2.00 | 5.00 | 2.50 | 2.50 | stable |
| 7 | 1:6.0 | 2.00 | 6.00 | 3.00 | 3.00 | stable |

The ingredients can be heated and mixed with sonication, ultrasonication, stirring, mixing, homogenization or external energy.

Manufacturing Process Number 6B):

All the ingredients were heated together. Vitamin E TPGS was combined with CBD Isolate, Essential Oil(s), and Carrier Oil(s), then the liquid emulsion was tested for stability at these ratios: (1:2.3) (1:3.0) (1:3.5) (1:4.0) (1:5.0) (1:6.0). CBD Isolate was measured, added to a beaker. Vitamin E TPGS was measured and added to the same beaker containing the CBD Isolate. Carrier Oil(s) was measured and added to the same beaker containing the Vitamin E TPGS, and CBD Isolate. Essential Oil(s) were measured and then added to the same beaker containing the Vitamin E TPGS, CBD Isolate, and Carrier Oil(s). The beaker with CBD Isolate, Vitamin E TPGS, Carrier Oil(s) and Essential Oil(s) were then placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, ratios (1:2.3) (1:3.0) (1:3.5) (1:4.0) (1:5.0) (1:6.0) did not separate, they are stable. After ten (10) months, these emulsion ratios did not separate, they were stable.

| | | Vitamin E | CBD | Essential | Carrier | |
|---|---|---|---|---|---|---|
| | | TPGS | Isolate | Oils | Oil | |
| Sample | Ratio | (g) wt | (g) wt | (g) wt | (g) wt | Stability |
| 1 | 1:2.3 | 3.00 | 3.45 | 1.73 | 1.73 | stable |
| 2 | 1:2.7 | 3.00 | 4.06 | 2.03 | 2.03 | stable |
| 3 | 1:3.0 | 3.00 | 4.50 | 2.25 | 2.25 | stable |
| 4 | 1:3.5 | 3.00 | 5.25 | 2.63 | 2.63 | stable |
| 5 | 1:4.0 | 2.00 | 4.00 | 2.00 | 2.00 | stable |
| 6 | 1:5.0 | 2.00 | 5.00 | 2.50 | 2.50 | stable |
| 7 | 1:6.0 | 2.00 | 6.00 | 3.00 | 3.00 | stable |

7) Test Emulsion Stability Using Carrier Oil(s).
Vitamin E TPGS, CBD Isolate, CoQ₁₀, were combined.
Two (2) different manufacturing processes were performed.

Manufacturing Process Number 7A:

Ingredients were heated separately and then slowly added together. Vitamin E TPGS was combined with different ratios of CBD Isolate, and CoQ₁₀, then the liquid emulsion was tested for stability at these ratios: (1:2.3) (1:3.0) (1:3.5) (1:4.0) (1:5.0) (1:6.0). Vitamin E TPGS was measured, added to a beaker, placed on a magnetic hot plate stirrer and heated between 40°−80° C. (104°−176° F.). CBD Isolate was measured, added to a beaker, placed on a magnetic hot plate stirrer and heated between 40°-80° C. (104°-176° F.). CoQ₁₀ was measured, added to a beaker, placed on a magnetic hot plate stirrer and heated between 40°-80° C. (104°-176° F.). The heated Vitamin E TPGS was stirred at 450 rpm for fifteen (15) minutes to ensure homogeneity of the liquid. The heated CBD Isolate was slowly added to the heated CoQ₁₀ and stirred for ten (10) minutes to ensure homogeneity of the liquid. The heated mixture of CBD Isolate and CoQ₁₀ were then added slowly to the beaker containing the heated Vitamin E TPGS. This melted combination of Vitamin E TPGS, CBD Isolate, and CoQ₁₀ (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Emulsion ratios (1:2.3) (1:3.0) (1:3.5) (1:4.0) (1:5.0) (1:6.0) separated within days or weeks, they are not stable.

| | | Vitamin E TPGS | CBD Isolate | CoQ₁₀ | |
|---|---|---|---|---|---|
| Sample | Ratio | (g) wt | (g) wt | (g) wt | Stability |
| 1 | 1:2.3 | 3.00 | 3.45 | 3.45 | unstable |
| 2 | 1:2.7 | 3.00 | 4.05 | 4.05 | unstable |
| 3 | 1:3.0 | 3.00 | 4.50 | 4.50 | unstable |
| 4 | 1:3.5 | 3.00 | 5.25 | 5.25 | unstable |
| 5 | 1:4.0 | 2.00 | 4.00 | 4.00 | unstable |
| 6 | 1:5.0 | 2.00 | 5.00 | 5.00 | unstable |
| 7 | 1:6.0 | 2.00 | 6.00 | 6.00 | unstable |

The ingredients can be heated and mixed with sonication, ultrasonication, stirring, mixing, homogenization or external energy.

Manufacturing Process Number 7B):

All the ingredients were heated together. Vitamin E TPGS was combined with CBD Isolate and CoQ₁₀, then the liquid emulsion was tested for stability at these ratios: (1:2.3) (1:3.0) (1:3.5) (1:4.0) (1:5.0) (1:6.0). CBD Isolate was measured, added to a beaker. Vitamin E TPGS was measured and added to the same beaker containing the CBD Isolate. CoQ₁₀ was measured and added to the same beaker containing the Vitamin E TPGS, and CBD Isolate. The beaker with CBD Isolate, Vitamin E TPGS, and CoQ₁₀ was then placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. The mixture/emulsions were allowed to sit at room temperature in a dark area and observed everyday for stability. Emulsion ratios (1:2.3) (1:3.0) (1:3.5) (1:4.0) (1:5.0) (1:6.0) separated within days or weeks, they are not stable.

| | | Vitamin E TPGS | CBD Isolate | CoQ₁₀ | |
|---|---|---|---|---|---|
| Sample | Ratio | (g) wt | (g) wt | (g) wt | Stability |
| 1 | 1:2.3 | 3.00 | 3.45 | 3.45 | unstable |
| 2 | 1:2.7 | 3.00 | 4.05 | 4.05 | unstable |
| 3 | 1:3.0 | 3.00 | 4.50 | 4.50 | unstable |
| 4 | 1:3.5 | 3.00 | 5.25 | 5.25 | unstable |
| 5 | 1:4.0 | 2.00 | 4.00 | 4.00 | unstable |
| 6 | 1:5.0 | 2.00 | 5.00 | 5.00 | unstable |
| 7 | 1:6.0 | 2.00 | 6.00 | 6.00 | unstable |

The ingredients can be heated and mixed with sonication, ultrasonication, stirring, mixing, homogenization or external energy.

Manufacturing Process Number 8A:

Ingredients were heated separately and then slowly added together. Vitamin E TPGS was combined with different ratios of CBD Isolate, Carrier Oil(s), and CoQ₁₀, then the liquid emulsion was tested for stability at these ratios: (1:2.3) (1:3.0) (1:3.5) (1:4.0) (1:5.0) (1:6.0). Vitamin E TPGS was measured, added to a beaker, placed on a magnetic hot plate stirrer and heated between 40°−80° C. (104°−176° F.). CBD Isolate was measured, added to a beaker, placed on a magnetic hot plate stirrer and heated between 40°-80° C. (104°-176° F.). The Carrier(s) Oil was measured, added to a beaker, placed on a magnetic hot plate stirrer and heated between 40°-80° C. (104°-176° F.). The CoQ₁₀ was measured, added to a beaker, placed on a magnetic hot plate stirrer and heated between 40°-80° C. (104°-176° F.). The heated Vitamin E TPGS was stirred at 450 rpm for fifteen (15) minutes to ensure homogeneity of the liquid. The heated Carrier Oil(s) were slowly added to the heated CoQ₁₀ and stirred for ten (10) minutes to ensure homogeneity of the liquid. The heated Carrier Oil(s) and CoQ₁₀ were slowly added to the heated CBD Isolate and stirred for fifteen (15) minutes to ensure homogeneity of the liquid. The heated mixture of CBD Isolate, Carrier Oil(s), and CoQ₁₀ was then added slowly to the beaker containing the heated Vitamin E TPGS. This melted combination of Vitamin E TPGS, CBD Isolate, Carrier Oil(s), and CoQ₁₀ (mixture) was stirred at 450 rpm for fifteen (15) minutes while the temperature was held at 70° C. (158° F.) to ensure homogeneity of the liquid. After stirring the mixture for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, emulsion ratios (1:2.3) (1:3.0) (1:3.5) (1:4.0) (1:5.0) (1:6.0) did not separate, they are stable. After ten (10) months, these emulsion ratios did not separate, they were stable.

| | | Vitamin E | CBD | | Carrier | |
|---|---|---|---|---|---|---|
| | | TPGS | Isolate | CoQ₁₀ | Oil | |
| Sample | Ratio | (g) wt | (g) wt | (g) wt | (g) wt | Stability |
| 1 | 1:2.3 | 3.00 | 3.45 | 1.73 | 1.73 | stable |
| 2 | 1:2.7 | 3.00 | 4.06 | 2.03 | 2.03 | stable |
| 3 | 1:3.0 | 3.00 | 4.50 | 2.25 | 2.25 | stable |
| 4 | 1:3.5 | 3.00 | 5.25 | 2.63 | 2.63 | stable |
| 5 | 1:4.0 | 2.00 | 4.00 | 2.00 | 2.00 | stable |
| 6 | 1:5.0 | 2.00 | 5.00 | 2.50 | 2.50 | stable |
| 7 | 1:6.0 | 2.00 | 6.00 | 3.00 | 3.00 | stable |

Manufacturing Process Number 8B):

All the ingredients were heated together. Vitamin E TPGS was combined with CBD Isolate, CoQ₁₀, and Carrier Oil(s), then the liquid emulsion was tested for stability at these ratios: (1:2.3) (1:3.0) (1:3.5) (1:4.0) (1:5.0) (1:6.0). CBD Isolate was measured, added to a beaker. Vitamin E TPGS was measured and added to the same beaker containing the CBD Isolate. Carrier Oil(s) was measured and added to the same beaker containing the Vitamin E TPGS, and CBD Isolate. CoQ₁₀ was measured and then added to the same beaker containing the Vitamin E TPGS, CBD Isolate, and Carrier Oil(s). The beaker with CBD Isolate, Vitamin E TPGS, Carrier Oil(s) and CoQ₁₀ were then placed on a magnetic hot plate stirrer and heated between 40°-80° C. (104°−176° F.). When the ingredients were melted, the mixture was then stirred at 450 rpm for fifteen (15) minutes with the temperature being held at 70° C. (158° F.) to ensure homogeneity of the liquid. After stirring for fifteen (15) minutes, the heat was turned off and the mixture continued to be stirred at 450 rpm until a temperature of 20° C. (68° F.) was achieved. The mixture/emulsions were allowed to sit at room temperature in a dark area and observed everyday for stability. Within one (1) week, ratios (1:2.3) (1:3.0) (1:3.5) (1:4.0) (1:5.0) (1:6.0) did not separate, they are stable. After ten (10) months, these emulsion ratios did not separate, they were stable.

| | | Vitamin E | | | Carrier | |
|---|---|---|---|---|---|---|
| | | TPGS | CBD | CoQ₁₀ | Oil | |
| Sample | Ratio | (g) wt | Isolate | (g) wt | (g) wt | Stability |
| 1 | 1:2.3 | 3.00 | 3.45 | 1.73 | 1.73 | stable |
| 2 | 1:2.7 | 3.00 | 4.06 | 2.03 | 2.03 | stable |
| 3 | 1:3.0 | 3.00 | 4.50 | 2.25 | 2.25 | stable |
| 4 | 1:3.5 | 3.00 | 5.25 | 2.63 | 2.63 | stable |
| 5 | 1:4.0 | 2.00 | 4.00 | 2.00 | 2.00 | stable |
| 6 | 1:5.0 | 2.00 | 5.00 | 2.50 | 2.50 | stable |
| 7 | 1:6.0 | 2.00 | 6.00 | 3.00 | 3.00 | stable |

Using certain ratios of Vitamin E TPGS with certain ratios of a Carrier Oil(s) with certain ratios of one or more Cannabinoid(s), full spectrum, broad spectrum, isolates, Terpenes, Vitamins, antioxidants, Essential Oils, etc., creates stable o/o emulsions, and increases topical and oral absorption/permeability and bioavailability. Being that Carrier Oils are all different in their chemical composition and viscosity, there are different ratios that are needed when combining Vitamin E TPGS with Carrier Oil(s) and other oils to create a stable o/o emulsion.

Testing has shown that Vitamin E TPGS can be used within a very limited range of ratios of CBD Isolate to create stable o/o emulsion(s). However, testing has further shown that Vitamin E TPGS can be used within a large range of ratios of CBD Isolate if a suitable amount of a Carrier Oil or oils are included, to create stable o/o emulsion(s). There are ratios within these ratios to create stable o/o emulsions. For example, in Manufacturing Process Number 6B, Sample 5, Vitamin E TPGS ratio is 1:4 to CBD Isolate, Essential Oils and Carrier Oil(s). The Essential Oils and Carrier Oil(s) are 2:1 to Vitamin E TPGS; this forms a stable o/o emulsion. Keeping the Vitamin E TPGS ratio at 1, change the CBD Isolate ratio to the other oils (Essential Oils and Carrier Oil) to 1:3 or 3:1 the o/o lipophilic emulsions will not be stable. The following examples will not be stable: 1:4 ratio of Vitamin E TPGS to all the other oils, the CBD Isolate having a 1:3 ratio to Essential Oils and Carrier Oil(s). Vitamin E TPGS 2g, CBD Isolate 2g, Essential Oils 3g, Carrier Oil 3g. Another example, 1:4 ratio of Vitamin E TPGS to all the other oils, the CBD Isolate having a 3:1 ratio to Essential Oils and Carrier Oil(s). Vitamin E TPGS 2g, CBD Isolate 6g, Essential Oils 1g, Carrier Oil(s) 1g.

Mixed heated/melted Vitamin E TPGS with a Cannabinoid (CBD Isolate) at ratios 1:1.7, 1:2.0, 1:2.3, 1:2.7, 1:3.0, 1:3.5, 1:4.0, 1:4.6, 1:1.5.0, 1:6.0 does not create a stable o/o liquid emulsion. Mixing heated/melted Vitamin E TPGS, Cannabinoid (CBD Isolate) and a Carrier Oil(s) at ratios 1:1.7, 1:2.0, 1:2.3, 1:2.7, 1:3.0, 1:3.5, 1:4.0, 1:4.6, 1:1.5.0, 1:6.0 creates a stable o/o liquid emulsion.

Mixing heated/melted Vitamin E TPGS, Cannabinoid (CBD Isolate) and Terpene at ratios 1:1.0, 1:2.0, 1:2.3, 1:2.7, 1:3.0, 1:3.5, 1:4.0, 1:4.6, 1:1.5.0 does not create a stable o/o liquid emulsion. Mixed heated/melted Vitamin E TPGS, Cannabinoid (CBD Isolate), Terpene, and a Carrier Oil(s) at ratios of 1:1.0, 1:2.0, 1:2.3, 1:2.7, 1:3.0, 1:3.5, 1:4.0, 1:4.6, 1:1.5.0 creates a stable o/o liquid emulsion.

Mixed heated/melted Vitamin E TPGS, Cannabinoid (CBD Isolate) and Essential Oil(s) at ratios of 1:2.3, 1:2.7, 1:3.0, 1:3.5, 1:4.0, 1:5.0, 1:1.60 does not create a stable o/o liquid emulsion. Mixed heated/melted Vitamin E TPGS, Cannabinoid (CBD Isolate), Essential Oil(s) and a Carrier Oil(s) at ratios of 1:2.3, 1:2.7, 1:3.0, 1:3.5, 1:4.0, 1:5.0, 1:1.60 creates a stable o/o liquid emulsion.

Mixed heated/melted Vitamin E TPGS, Cannabinoid (CBD Isolate) and CoQ₁₀ which is a lipophilic Vitamin/antioxidant at ratios of 1:2.3, 1:2.7, 1:3.0, 1:3.5, 1:4.0, 1:5.0, 1:1.60 does not create a stable o/o liquid emulsion. Mixed heated/melted Vitamin E TPGS, Cannabinoid (CBD Isolate) and CoQ₁₀ which is a lipophilic Vitamin/antioxidant and a Carrier Oil(s) at ratios of 1:2.3, 1:2.7, 1:3.0, 1:3.5, 1:4.0, 1:5.0, 1:1.60 creates a stable o/o liquid emulsion.

As used in this application, the words “a,” “an,” and “one” are defined to include one or more of the referenced item unless specifically stated otherwise. The terms “approximately” and “about” are defined to mean+/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A Cannabinoid emulsion composition comprising:
Vitamin E TPGS, a Cannabinoid, and a Carrier Oil, in the absence of water, heated and mixed in such proportions that the composition forms nano or micro sized micelles when ingested.

2. A method for forming a Cannabinoid emulsion composition, the method comprising the steps of:
providing Vitamin E TPGS, a Cannabinoid, and a Carrier Oil;
heating the Vitamin E TPGS, a Cannabinoid, and a Carrier Oil to a temperature between 30-100 degrees Celsius (86-212 degrees Fahrenheit), while mixing; and
allowing the second composition to cool, while mixing, until an emulsion is formed.

3. The method of claim 2, wherein mixing is performed at between 200-1000 rotations per minute.

4. The method of claim 2, wherein the Carrier Oil is high in long chain triglycerides (LCT) and includes at least 5% polyunsaturated fatty acids and monounsaturated fatty acids.

5. The method of claim 2, wherein the Carrier Oil is high in long chain triglycerides (LCT).

6. The method of claim 2, wherein the Carrier Oil is high in medium chain triglycerides (MCT).

7. The method of claim 2, wherein the Carrier Oil includes at least 5% polyunsaturated fatty acids and monounsaturated fatty acids.

8. The method of claim 2, wherein the composition is heated to a temperature of between 30-100 degrees Celsius (86-212 degrees Fahrenheit).

9. The method of claim 2, wherein the composition further includes at least one Terpene.

10. The method of claim 9, wherein the at least one Terpene includes one of the following: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

11. The method of claim 2, wherein the Cannabinoid includes at least one of the following: THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, or THC isolate.

12. The method of claim 2, wherein the Carrier Oil includes at least one of the following: Acai palm oil, Almond oil, Apricot oil, Argan oil, Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil, Brazil nut oil, Buriti oil, Calendula oil, Camellia seed oil, Canola, Carapa oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Emu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil, Kukui nut oil, Linseed oil, Macadamia oil, Manketti nut oil, Marula oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil, Rice bran oil, Rosehip oil, Safflower, Sea Buckthorn oil, Safflower oil, Salicornia oil, Sesame seed oil, Solarium oil, Soybean oil, Strawberry seed oil, Sunflower seed oil, St John's Wort Oil, Sweet Almond oil, Tamanu oil, Tomato seed oil, Trauma oil, Vegetable oil, Vigna mungo oil, Walnut oil, Watermelon seed oil, and Wheat germ oil.

13. The method of claim 2, wherein the composition further includes at least one Essential Oil.

14. The method of claim 13, wherein the Essential Oil includes at least one of the following: Black Pepper Essential Oil, Clove Essential Oil, Peppermint Essential Oil, lemongrass Essential Oil. In some embodiments, the Cannabis Oil composition includes one or more added Essential Oils including but not limited to the following: Sweet Orange (Citrus sinensis spp), Peppermint (Mentha piperita spp), Lemon (Citrus limon spp), Lavender (Lavendula angustifolia spp) and Vanilla (Vanilla planifolia spp), Agarwood; Agarwood Attar; Arnica; Ahibero; Allspice; Almond, bitter; Amber Oil; Ambrette Seed; Amyris; Angelica Root; Angelica Seed; Aniseed; Anise; Anise (star); Armoise (Mugwort); Artemisia vestita; Asafoetida; Bakul; Balsam of Peru Oil; Balsam of Peru Resin; Balsamite; Baobab Oil; Basil, Sweet ct Linalool; Basil, Sweet ct Linalool—Organic; Basil, Sweet ct Methyl Chavicol—Organic; Bay; Beeswax; Bergamot; Birch; Boldo; Boronia; Black Cumin; Black Currant Bud; Black Pepper; Blue Lotus Attar; Broom; Buchu; Bupleurum (Bupleurum fruticosum); Buddha wood; Butter; Cabreuva; Cade; Cajuput; Calamus; Calendula; Camomile (or Chamomile); Camphor; Cananga; Cangerana; Cape Chamomile (Ericephalus punctulatus) S. Africa, Wild Harvest; Cape May; Caraway; Caraway; Cardamom; Carnation; Carrot Seed; Cascarilla; Cassia; Cassie; Catnip; Cedar (Cedrus) India; Cedarwood; Cedarwood, Atlas—Organic; Cedarwood, Himalayan; Cedarwood, Texas; Cedarwood, Virginia; Celery leaf, Celery Seed; Chamomile, Blue; Chamomile; Chamomile, Roman (Anthemis nobilis); Champa Attar (Michelia champaca) India; Champaca; Chaste tree; Cilantro; Cinnamon; Cinnamon Bark; Cistus; Cistus (Cistus ladaniferus) Corsica; Citronella; Clary Sage Absolute; Clary Sage, Bulgaria; Clary Sage, Russia; Clary Sage, USA; Clementine; Clove; Clove Bud; Cacao; Coconut Pulp; Coffee Bean Oil; Cognac, Green; Coleus; Combava (fruit or leaf); Copaiba; Coriander; Coriander Seed; Cucumber Hydrosol; Cumin; Cumin Seed; Cypress Leaf, Cypress, Blue; Davana; Dill; Elemi; Eucalyptus, Blue Gum; Eucalyptus, Blue Mallee; Eucalyptus, Lemon; Fennel (Foeniculum vulgare) Bulgaria; Fennel, Sweet; Fenugreek; Fern (sweet); Fleabane; Fir Needle; Fir, Balsam; Fir, Douglas; Fir, Silver; Fragonia; Frankincense, India; Frankincense, Somalia; Frankincense Frereana; Frankincense, Oman; Frankincense, Oman; Frankincense, Somalia; Galangal; Galbanum; Geranium; Geranium, Egypt; Geranium, Rose; Geranium, South Africa; Ghandi root; Ginger; Ginger Lily; Ginger, Fresh; Gingergrass (Cymbopogon martinii); Goldenrod; Grapefruit, Pink; Grapefruit, Ruby Red; Grapefruit, White; Hay; Helichrysum, Albania; Helichrysum, Croatia; Hina Attar, India; Hop; Hyssop Decumbens; Hyssop; Immortelle; Jasmine Absolute, Egypt; Jasmine Absolute, India; Jasmine Concrete; Jasmine; Jasmine Sambac; Jatamansi, (Nardostachs jatamansi) Juniper; Juniper Berry (Juniperus communis) or leaf, Kaffir Lime; Kava Kava; Labdanum; Larch needle; Laurel (Laurus nobilis) Corsica; Laurel Leaf, Lavandin, Grosso; Lavender—High Elevation; Lavender—Wild; Lavender Absolute; Lavender Hydrosol; Lavender, Bulgaria; Lavender, France; Lavender, Maillette; Leleshwa; Lemon; Lemon Tea Tree; Lemon verbena; Lemongrass; Lentisque (Pistacia lentiscus) Corsica; Lime; Lime Essence Oil; Lime, Distilled; Liquidambar (Styrax); Longoza; Lotus Absolute, Pink; Lotus Absolute, White; Lovage leaf; Lovage root; Magnolia flower; Mandarin; Mandarin, Green; Mandarin, Red; Mandarin, Yellow; Mango ginger; Marjoram; Manila oil; Melissa; Mint; Mint, Himalayan (Mentha arvensis); Mitti Attar; Motia Attar (Jasmine sambac) India; Mugwort; Mustard; Myrrh; Myrtle, Green; Myrtle (Myrtus Communis); Nagarmotha (Cypriol); Neem (Azadirachta indicd) India; Neroli; Niaouli; Nutmeg; Nut grass; Oakmoss Absolute; Oakwood; Opopanax, Sweet Myrrh (Commiphora guidotti); Orange, Blood; Orange, Sweet; Orange, Wild; Orange Blossom; Orange Essence Oil; Orange, Bitter Green; Orange, Bitter Red; Oregano; Orris Butter; Osmanthus Absolute; Palmarosa; Palmarosa, Nepal; Palmarosa, Sri Lanka; Palo Santo (Bursera graveolens); Palo Santo; Patchouli; Absolute; Patchouli, Dark; Patchouli, Light; Patchouli, Sri Lanka; Pennyroyal; Pepper, Black; Peppercorn, Pink; Peppermint, Chocolate; Peppermint, France; Petitgrain Absolute; Petitgrain Bigarade; Petitgrain sur Fleurs; Petitgrain, Mandarin; Pimento; Pine; Pinion Juniper Co-distillation, Colorado, Wild Harvest; Pinon Pine (Pinus edulis) Colorado, Wild Harvest; Pitta blend (Lavender, Rose Geranium, Ruh Khus); Plai; Pomegranate Seed; Rhododendron (Rhododendron anthopogon); Rhododendron Leaf, Rosalina; Rose; Rose Attar; Rose de Mai Absolute; Rose de Mai Concrete; Rose de Mai Organic Extract; Rose geranium; Rose Hip Seed; Rose Otto, Bulgaria; Rose Otto, Turkey; Rose Otto, White—Organic; Rose vetiver; Rosemary Antioxidant; Rosemary ct Cineole; Rosemary ct Verbenone; Rosewood; Rue; Ruh Khus (Vetiveria zizaniodes); Saffron Attar, India; Sage; Samphire (Cristhmum maritimum) Corsica; Sandalwood; Sandalwood, New Caledonia; Sandalwood, Australian—Premium; Sandalwood (Santalum spicatum), Australia; Sandalwood Oil, Royal Hawaiian (Santalum paniculatum); Sandalwood, Royal Hawaiian; Sassafras; Savitri Rose Perfume; Sea Buckthorn; Seaweed; Sierra Juniper (Juniperus occidentalis); Spearmint; Spearmint (Mentha Spicatd) Israel; Spikenard; Spikenard, Green; Spruce, Black; Spruce (Picea mariand) Canada; St. John's Wort (Hy peri cum perforatum) Bulgaria; Tagetes; Tamanu (Foraha) Oil; Tangelo; Tangerine; Tangerine Murcott; Tansy; Tansy, Blue; Tarragon; Tea Tree; Tea Tree (Leptospermum citratum), Lemon Scented; Tea Tree (Melaleuca alternifolid) South Africa; Thuja; Thyme; Thyme ct Linalool; Tobacco; Tonka Bean; Tuberose; Tulsi, Holy Basic Oil (Ocimum sanctum), Turmeric; Vanilla; Vanilla Bourbon; Verbena; Vetiver—Double Distilled; Vetiver, El Salvador; Vetiver, Haiti; Vetiver, Sri Lanka; Violet Leaf, White Fir (Abies concolor), White Lotus Attar; White Sage (Salvia apiana), Wild Carrot, Corsica; Wintergreen; Wintergreen; Yarrow; Yarrow, Blue; Ylang Ylang; and Yuzu.

15. A method for forming a Cannabinoid emulsion composition, the method comprising the steps of:
providing Vitamin E TPGS, a Cannabinoid, and a Carrier Oil that includes at least 5% polyunsaturated fatty acids and monounsaturated fatty acids;
adding the Vitamin E TPGS, the Cannabinoid, and the Carrier Oil, forming the composition;
heating the composition to a temperature between 30-100 degrees Celsius (86-212 degrees Fahrenheit), while mixing; and
allowing the composition to cool, while mixing, until the composition is at room temperature and an emulsion is formed.

16. The method of claim 15, wherein mixing is performed at between 200-1000 rotations per minute.

17. The method of claim 15, wherein the composition further includes at least one Terpene.

18. The method of claim 17, wherein the at least one Terpene includes one of the following: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

19. The method of claim 15, wherein the Cannabinoid includes at least one of the following: THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), CBD isolate, full spectrum CBD, broad spectrum CBD, THC distillate, THC isolate

20. The method of claim 15, wherein the Carrier Oil includes at least one of the following: Acai palm oil, Almond oil, Apricot oil, Argan oil, Arnica oil, Avocado oil, Babassu oil, Barbary Fig Seed oil, Baobab oil, Black Cumin Seed oil, Black Currant seed oil, Black Raspberry seed oil, Blackberry seed oil, Blueberry seed oil, Borage seed oil, Brazil nut oil, Buriti oil, Calendula oil, Camellia seed oil, Canola, Carapa oil, Carrot seed oil, Cashew oil, Castor oil, Chardonnay Grape seed oil, Cherry Kernel oil, Chia seed oil, Cloudberry seed oil, Cocoa butter oil, Coconut oil, Corn Oil, Cottonseed oil, Cranberry seed oil, Cucumber seed oil, Elderberry seed oil, Emu oil, Evening primrose oil, Fenugreek oil, Flaxseed/Linseed, Goji Berry seed oil, Grape seed oil, Graviola oil, Guava seed oil, Hazelnut oil, Hemp seed oil, Jambu oil, Jojoba oil, Kukui nut oil, Linseed oil, Macadamia oil, Manketti nut oil, Marula oil, Meadowfoam seed oil, Melon seed oil, Milk Thistle seed oil, Moringa oil, Mustard oil, Neem oil, Olive oil, Palm oil, Passion fruit oil, Peach oil, Peanut oil, Pecan oil, Perilla oil, Pistachio oil, Plum Kernel oil, Pomegranate oil, Poppyseed oil, Pracaxi oil, Prickly Pear seed oil or Barbary Fig, Pumpkin seed oil, Red Raspberry seed oil, Rice bran oil, Rosehip oil, Safflower, Sea Buckthorn oil, Safflower oil, Salicornia oil, Sesame seed oil, Solarium oil, Soybean oil, Strawberry seed oil, Sunflower seed oil, St John's Wort Oil, Sweet Almond oil, Tamanu oil, Tomato seed oil, Trauma oil, Vegetable oil, Vigna mungo oil, Walnut oil, Watermelon seed oil, and Wheat germ oil.
